SEQUENCE LISTING (6 sequences)

SEQ ID NO: 1 - protein, 188 aa, Nocardioides sp. NRRL 18262
ADIIGGLAYTMGGRCSVGFAATNAAGQPGFVTAGHCGRVGTQVTIGNGRGVFEQSVFPGNDAAFVRGTSNFTLTNLVSRYNTGGYAAVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 2 - protein, 17 aa, Nocardiopsis alba
ADIIGGLAYTMGGRCSV

SEQ ID NO: 3 - protein, 314 aa, Bacillus horneckiae
  mat_peptide (1)..(314)
EVTATPSTQTPWGIKSIYNDQSITKTTGGSGIKVAVLDTGVHTGHIDLAGSSEQCKDFTQSNPLVNGSCTDRQGHGTHVAGTVLAHGGSDGQGVYGVAPQAKLWAYKVLGDNGSGYSDDIAAAIRHVADEASRTGSKVVINMSLGSSGKDSLIASAVDYAYGKGVLIVAAAGNSGSGSNTIGYPAALVNAVAVAALENVQQNGTYRVANFSSRGNPATAGDFRIQERDVEVSAPGASVESTWYNGGYNTISGTSMATPHVAGLAAKIWSSNSSLSHSQLRTELQNRAKVYDIKGGIGAGTGDDYASGFGYPRVK

SEQ ID NO: 4 - protein, 311 aa, Bacillus sp. [sequence omitted]

SEQ ID NO: 5 - protein, 311 aa, Artificial Sequence
  Protein Engineered variant
  mat_peptide (1)..(311)
AVPSTQTPWGIKSIYNDQSITKTTGGKGIKVAVLDTGVYTSHLDLAGSAEQCKDFTQSNPLVDGSCTDRQGHGTHVAGTVLAHGGSNGQGVYGVAPQAKLWAYKVLGDKGEGYSDDIAAAIRHVADEASRTGSKVVINMSLGSSAKDSLIASAVDYAYGKGVLIVAAAGNEGPKPNTIGYPAGFVNAVAVAALENVQEKGTYRVADFSSRGNPATAGDYIIQERDIEVSAPGASVESTWYTGGYNTISGTSMATPHVAGLAAKIWSANTSLSHSQLRTELQNRAKVYDIKGGIGAGPGDDYASGFGYPRVK

SEQ ID NO: 6 - protein, 12 aa, Artificial Sequence
  Conserved motif TGXKV[I/V]XXMSLG.
  MISC_FEATURE (3)..(3): The amino acid in position 3 is any amino acid.
  MISC_FEATURE (6)..(6): The amino acid in position 3 is valine (V) or isoleucine (I).
  MISC_FEATURE (7)..(7): The amino acid in position 7 is any amino acid.
  MISC_FEATURE (8)..(8): The amino acid in position 8 is any amino acid.
TGXKVXXXMSLG

(19) United States
(12) Patent Application Publication
(10) Pub. No.: US 2025/0318552 A1
(43) Pub. Date: Oct. 16, 2025

(54) METHOD OF IMPROVING CARBOHYDRATE DIGESTIBILITY BY A CARBOHYDRATASE IN AN ANIMAL FEED BY EMPLOYING SERINE PROTEASE

(71) Applicants: DSM IP ASSETS B.V., HEERLEN (NL); NOVOZYMES A/S, Bagsvaerd (DK)

(72) Inventors: Aaron COWIESON, Kaiseraugst (CH); Eduardo DELLA PIA, Bagsvaerd (DK); Jose-Otavio SORBARA, Kaiseraugst (CH)

(21) Appl. No.: 18/546,866

(22) PCT Filed: Feb. 28, 2022
(86) PCT No.: PCT/EP2022/025071

§ 371 Date: Aug. 17, 2023

(30) Foreign Application Priority Data
Feb. 26, 2021 (CH) 00209/21
Feb. 26, 2021 (DK) PA202100209

(51) Int. Cl. A23K 20/189; A23K 10/30

(52) U.S. Cl. CPC A23K 20/189; A23K 10/30; C12Y 302/01008; C12Y 302/01015; C12Y 302/0102; C12Y 302/01073; C12Y 304/21

(57) ABSTRACT

The present invention provides a method for improving the digestibility of a carbohydrate by a carbohydrase in an animal feed such as soybean-meal, corn, wheat, wheat middlings, oats, rye, barley and sorghum; and/or roughages such as hay and pasture plants, or mixture thereof by adding one or more proteases, preferably a serine protease such as a Nocardiopsis serine protease or a S8 serine protease. The present invention also provides a feed composition suitable for the above methods.

### TECHNICAL FIELD

The present invention is related to a method for boosting the activity of a carbohydrase in an animal feed and/or improving digestibility of a carbohydrate in an animal.

### BACKGROUND OF THE INVENTION

Carbohydrates are energy-providing feed components composed of carbon, hydrogen and oxygen. They should make up about 75 percent of an animal's diet. The energy they provide powers muscular movements. Carbohydrates also produce the body heat that helps keep the animal warm. They aid in the use of proteins and fats by the body. Carbohydrates are not stored in the body. They must be provided in the animal's diet every day. Unused carbohydrates are converted into fats to be stored.

Carbohydrates can be classified into two categories: storage carbohydrates and structural carbohydrates. Storage carbohydrates include starch and simple sugars, such as fructose and saccharose. These carbohydrates, along with lipids in the embryonic part of seeds, are the main energy sources for the new plants that will be emerging from cereal seeds.

Structural carbohydrates, on the other hand, including the well-known non-starch polysaccharides, are responsible for cellular form and structure, and are located mostly in the outer cellular membrane. Structural carbohydrates, commonly referred to as “fiber,” are hardly digested by monogastric animals due to lack of suitable endogenous enzymes. Thus, the majority of structural carbohydrates are fermented in the hind gut, where they may release limited useful energy levels.

Carbohydrases are specific commercial enzyme preparations that attack carbohydrates releasing energy that would be otherwise lost for the animal. They work mainly by opening the cell wall structure of intact plant cells, release thus not only energy (starch), but also other nutrients, such as protein, minerals and lipids. In addition, plant cell wall fractions increase intestinal viscosity that leads to reduced nutrient absorption, accelerated proliferation of pathogens, such as Escherichia coli, and other problems, such as sticky droppings and dirty eggs. Today carbohydrases are becoming increasingly popular in animal feeds.

Surprisingly, it is discovered that proteases can boost the activity of carbohydrases to hydrolyze carbohydrates in an animal feed and thus potentially improving digestibility of carbohydrates in an animal.

### SUMMARY OF THE INVENTION

Accordingly, the present invention provides a method for boosting the activity of a carbohydrase or improving hydrolyzation of a carbohydrate by a carbohydrase in an animal feed, and a method for improving digestibility of a carbohydrate in an animal by using one or more proteolytic enzymes, i.e., proteases.

The present invention also provides a feed composition comprising one or more proteolytic enzymes, i.e., proteases and a carbohydrase for improving hydrolyzation of carbohydrates in an animal feed and/or for improving digestibility of a carbohydrate in an animal and use thereof.

### DETAILED DESCRIPTION OF THE INVENTION

In the present invention, the term “animal” or “animals” refers to any animal except humans. Examples of animals are monogastric animals, including but not limited to pigs or swine (including but not limited to piglets, growing pigs and sows); poultry such as turkeys, ducks, quail, guinea fowl, geese, pigeons (including squabs) and chicken (including but not limited to broiler chickens (referred to herein as broilers), chicks, layer hens (referred to herein as layers)); pets such as cats and dogs; horses (including but not limited to hotbloods, coldbloods and warm bloods), crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

In the present invention, the term “animal feed”, “animal diet” or “feed” refers to any compound, preparation, or mixture suitable for or intended for intake by an animal and capable of maintaining life and/or promoting production of the animal without any additional substance being consumed except water. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

In the present invention, the term “concentrates” refers to feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

In the present invention, the term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

In the present invention, the term “roughage” refers to dry plant material with high levels of fiber, such as fiber, bran, husks from seeds and grains and crop residues (such as stover, copra, straw, chaff, sugar beet waste).

In the present invention, the term “animal feed additive” refers to an ingredient or combination of ingredients added to the animal feed, usually used in micro quantities and requires careful handling and mixing. Such ingredient includes but is not limited to vitamins, amino acids, minerals, enzymes, eubiotics, colouring agents, growth improving additives and aroma compounds/flavourings, polyunsaturated fatty acids (PUFAs); reactive oxygen generating species, antioxidants, anti-microbial peptides, anti-fungal polypeptides and mycotoxin management compounds etc.

In the present invention, the term “hydrolyzation” refers to the carbohydrates to be hydrolysed are broken down under acidic or alkaline conditions and/or in the presence of an enzyme into soluble sugar molecules. For example, a polysaccharide can be hydrolysed into monosaccharaides or disaccharides or oligosaccharides by treating with concentrated hydrochloric acid, and starch can be broken into maltose by treating with concentrated hydrochloric acid or an amylase. The hydrolyzation of carbohydrates is improved when more carbohydrates, for example, 1%, 2%, 3%, 4%, 5% or more carbohydrates, are broken down into soluble sugar molecules in the presence of the feed composition according to the present invention compared to a feed composition otherwise.

In the first aspect, the present invention provides a method for boosting the activity of a carbohydrase in an animal feed comprising adding to the animal feed one or more proteolytic enzymes, i.e., proteases.

Continues to the first aspect, the present invention also provides a method for improving hydrolyzation of a carbohydrate in an animal feed comprising adding to the animal feed one or more proteolytic enzymes, i.e., proteases, and a carbohydrase.

Continues to the first aspect, the present invention further provides a method for improving digestibility of a carbohydrate in an animal comprising administering to the animal one or more proteolytic enzymes, i.e., proteases, and a carbohydrase in an animal feed.

In the present invention, proteolytic enzymes or proteases catabolize peptide bonds in proteins breaking them down into fragments of amino acid chains, or peptides.

Proteases are classified on the basis of their catalytic mechanism into the following groups: serine proteases(S), cysteine proteases (C), aspartic proteases (A), metalloproteases (M), and unknown, or as yet unclassified, proteases (U) (see Handbook of Proteolytic Enzymes, A. J. Barrett, N. D. Rawlings, J. F. Woessner (eds), Academic Press (1998)). The protease according to the present invention is a serine protease, preferably an acid stable serine protease, and more preferably a S8 protease.

There are no limitations on the origin of the protease for use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by site-directed mutagenesis, by PCR (using a PCR fragment containing the desired mutation as one of the primers in the PCR reactions), or by random mutagenesis. The preparation of consensus proteins is described in e. g. EP 0 897 985.

Preferably, the protease according to the present invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i.e. the term covers the expression of wild-type, naturally occurring proteases, as well as expression in any host of recombinant, genetically engineered or synthetic proteases.

Examples of microorganisms are bacteria, e. g. bacteria of the phylum Actinobacteria phy. nov., e. g. of class I: Actinobacteria, e. g. of the Subclass V: Actinobacteridae, e. g. of the Order I: Actinomycetales, e. g. of the Suborder XII: Streptosporangineae, e. g. of the Family II: Nocardiopsaceae, e. g. of the Genus I: Nocardiopsis, e. g. Nocardiopsis sp. NRRL 18262, and Nocardiopsis alba; e.g. of the species Bacillus or mutants or variants thereof exhibiting protease activity. This taxonomy is on the basis of Berge's Manual of Systematic Bacteriology, 2nd edition, 2000, Springer (preprint: Road Map to Bergey's).

Further examples of microorganisms are fungi, such as yeast or filamentous fungi.

Preferred protease according to the present invention is an acid stable serine protease obtained or obtainable from the Genus: Nocardiopsis, such as those derived from Nocardiopsis dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2); or the Genus: Bacillus, e.g. Bacillus horneckiae, Bacillus sp TY145, Bacillus sp-13380, Bacillus idriensis, Bacillus sp-62451 and Bacillus oceanisediminis; as well as homologous proteases.

There are no limitations on the origin of the acid stable serine protease for use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by Random Mutagenesis. The preparation of consensus proteins is described in e. g. EP 0 897 985.

Examples of acid-stable proteases for use according to the invention are
a) the proteases derived from Nocardiopsis sp. NRRL 18262, and Nocardiopsis alba;
b) proteases of at least 60, 65, 70, 75, 80, 85, 90, or at least 95% amino acid identity to any of the proteases of (i);
c) proteases of at least 60, 65, 70, 75, 80, 85, 90, or at least 95% identity to any of SEQ ID NO: 1, and/or SEQ ID NO: 2.

For calculating percentage identity, any computer program known in the art can be used. Examples of such computer programs are the Clustal V algorithm (Higgins, D. G., and Sharp, P. M. (1989), Gene (Amsterdam), 73, 237-244; and the GAP program provided in the GCG version 8 program package (Program Manual for the Wisconsin Package, Version 8, Genetics Computer Group, 575 Science Drive, Madison, Wisconsin, USA 53711) (Needleman, S. B. and Wunsch, C. D., (1970), Journal of Molecular Biology, 48, 443-453.

The protease for use according to the invention can be produced or expressed in the original wild-type microbial strain, in another microbial strain, or in a plant; i. e. the term covers the expression of wild-type, naturally occurring proteases, as well as expression in any host of recombinant, genetically engineered or synthetic proteases.

In the present context, the term acid-stable means, that the protease activity of the pure protease enzyme, in a dilution corresponding to A280=1.0, and following incubation for 2 hours at 37 C in the following buffer:
100 mM succinic acid, 100 mM HEPES, 100 mM CHES,
100 mM CABS, 1 mM CaCl2, 150 mM KCl, 0.01% Triton [symbol] X-100, pH 3.5,
   is at least 40% of the reference activity, as measured using the assay described in Example 1 herein (substrate: Suc-AAPF-pNA, pH 9.0, 25° C.).

In particular embodiments of the above acid-stability definition, the protease activity is at least 45, 50, 55, 60, 65, 70, 75, 80, 85, 90, 95, or at least 97% of the reference activity.

The term reference activity refers to the protease activity of the same protease, following incubation in pure form, in a dilution corresponding to A280=1.0, for 2 hours at 5 C in the following buffer: 100 mM succinic acid, 100 mM HEPES, 100 mM CHES, 100 mM CABS, 1 mM CaCl2), 150 mM KCl, 0.01% Triton [symbol] X-100, pH 9.0, wherein the activity is determined as described above.

In other words, the method of determining acid-stability comprises the following steps:
a) The protease sample to be tested (in pure form, A280=1.0) is divided in two aliquots (I and II);
b) Aliquot I is incubated for 2 hours at 37° C. and pH 3.5;
c) Residual activity of aliquot I is measured (pH 9.0 and 25° C.);
d) Aliquot II is incubated for 2 hours at 5° C. and pH 9.0;
e) Residual activity of aliquot II is measured (pH 9.0 and 25° C.);
f) Percentage residual activity of aliquot I relative to residual activity of aliquot II is calculated.

Alternatively, in the above definition of acid stability, the step b) buffer pH-value may be 1.0, 1.5, 2.0, 2.5, 3.0, 3.1, 3.2, 3.3, or 3.4.

In other alternative embodiments of the above acid stability definition relating to the above alternative step b) buffer pH-values, the residual protease activity as compared to the reference, is at least 5, 10, 15, 20, 25, 30, 35, 40, 45, 50, 55, 60, 65, 70, 75, 80, 85, 90, 95, or at least 97%.

In alternative embodiments, pH values of 6.0, 6.5, 7.0, 7.5, 8.0, or 8.5 can be applied for the step d) buffer.

In the above acid-stability definition, the term A280=1.0 means such concentration (dilution) of said pure protease which gives rise to an absorption of 1.0 at 280 nm in a 1 cm path length cuvette relative to a buffer blank.

And in the above acid-stability definition, the term pure protease refers to a sample with a A280/A260 ratio above or equal to 1.70.

In another particular embodiment, the protease for use according to the invention, besides being acid-stable, is also thermostable.

The term thermostable means one or more of the following: That the temperature optimum is at least 50° C., 52° C., 54° C., 56° C., 58° C., 60° C., 62° C., 64° C., 66° C., °68 C, or at least °70 C.

Another preferred protease according to the present invention is a protease defined by polypeptides having S8 protease activity, wherein the polypeptide is selected from the list consisting of: a) a polypeptide having a sequence identity of at least 70% to any one of SEQ ID NOs 3-6;
b) a variant of any one of SEQ ID NOs: 3-6, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
c) a polypeptide comprising the polypeptide of (a′) or (b′) and a N-terminal and/or C-terminal His-tag and/or HQ-tag;
d) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and
e) a fragment of the polypeptide of (a′) or (b′) having protease activity and having at least 90% of the length of the mature polypeptide

Commercially available proteases which are covered by definitions above are Ronozyme®ProAct (DSM Nutritional Products AG, Switzerland), Ronozyme®ProAct360 (DSM Nutritional Products Ltd., Switzerland), AxtraPro (subtilisin-type protease, Dupont, USA), Poultrygrow (mixture of different proteases, Jefo, USA), Cibenza DP100 (Novus, USA).

In a preferred example, the intended dosage of the protease is 0.01-200 mg protease enzyme protein per kg final feed.

According to the present invention, the protease may be provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed. One protease unit (PROT) is the amount of enzyme that releases 1 μmol of p-nitroaniline (pNA) from 1 mM substrate (such as N-succinyl-Ala-Ala-Pro-Phe-pNA) per minute at pH 9.0 and 37° C.

In the present invention, the carbohydrase may be any carbohydrase which can be added into an animal feed for feeding an animal. Examples of carbohydrases include but are not limited to hemicellulases, pectinases, glucanases, amylases (such as α-amylase, β-amylase and γ-amylase), xylanases, galactosidases maltases and mixtures thereof.

Preferred xylanases are 1,4-ß-xylanases, for example endo-1,4-β-xylanases produced by Trichoderma reesei. Examples of carbohydrase mixtures are mixtures of different xylanases or carbohydrase blends comprising at least two enzymes selected from the group consisting of xylanases, glucanases, hemicellulases, pectinases, amylases, galactosidases, maltases. Mixtures containing a combination of more than 10 active enzymes can be produced by only one non-genetically modified fungus, as for example Talaromyces versatilis.

Commercially available carbohydrases which are covered by the definitions above and can be boosted by proteases according to the invention are Ronozyme®VP (Glucanase, DSM Nutritional Products AG, Switzerland), Ronozyme®WX (Xylanase, DSM Nutritional Products Ltd., Switzerland), RONOZYME® HiStarch (Amylase from Bacillus licheniformis, DSM Nutritional Products AG, Switzerland), Rovabio (Carbohydrase blend, Adisseo, France), ECONASE® (Xylanase, AB Enzymes, Germany), CIBENZA CSM (mixture of xylanase, β-glucanase and galactosidase, Novus International, USA) and AllzymeSSF (Carbohydrase blend, Alltech USA).

According to the present invention, the carbohydrase is provided in a dosage of between 10 units/kg animal feed and 5,000 units/kg animal feed, for example in one of the following: 10, 20, 40, 50, 60, 80, 100, 200, 500, 800, 1,000, 2,000, 3,000, 4,000 and 5,000 units/kg animal feed.

In the present invention, the activity of a carbohydrase can be represented by hydrolyzation of a carbohydrate by the carbohydrase, which can be easily measured by a process known in the art, for example, by testing the hydrolysate of the carbohydrate treated by the carbohydrase as shown in the examples of the present application. The activity of a carbohydrase is boosted when the carbohydrate breaks more carbohydrate, such as 1%, 2%, 3%, 4%, 5% or more, into soluble sugar molecules in the presence of a protease compared to the same conditions without the protease.

In the present invention, the activity of a carbohydrase may be boosted or the hydrolyzation of carbohydrates may be improved by 1%, 2%, 3%, 4%, 5%, 10%, 15%, 20% or more based on the amount of the hydrolysate obtained from the hydrolyzation with the carbohydrase.

As known in the art, carbohydrates are the basic source of energy for all animals and must be provided in the animal diets every day. The carbohydrates suitable for the animal feed according to the present invention may be monosaccharaides such as galactose, glucose, fructose, ribose, arabinoses and xylose; disaccharides such as maltose, sucrose and lactose; oligosaccharides such as arabinoxylans; and/or polysaccharides such as starch including amylose, amylopectin and modified starches, non-starch polysaccharides (NSP) including pentosans, glycogen, fibre, cellulose, hemicellulose, chitin, pectin and/or hydrocolloids.

In the present invention, the carbohydrates to be hydrolysed by the carbohydrase may be oligosaccharides and/or polysaccharides. Preferably, the carbohydrates are arabinoxylans, starch or NSP such as fibre, cellulose, hemicellulose and pectin.

In the present invention, the carbohydrates may be in the forms of concentrates such as soy-bean meal, corn, wheat, wheat middlings, oats, rye, barley and sorghum; and/or roughages such as hay and pasture plants, or mixture thereof. Preferably, the carbohydrates in the animal feed are in the forms of soy-bean meal, corn, wheat, wheat middlings, oats, rye or barley, or mixture thereof.

Preferably, the animal feed according to the present invention is an animal diet based on soy-bean meal, corn and/or wheat.

As anticipated by any person skilled in the art, the animal feed according to the present may also include micro-ingredients.

The micro-ingredients include but are not limited to aroma compounds; antimicrobial peptides; polyunsaturated fatty acids (PUFAs); reactive oxygen generating species; at least one enzyme, and fat- and water-soluble vitamins, as well as minerals.

Examples of antimicrobial peptides (AMP's) are CAP18, leucocin A, protegrin-1, thanatin, defensin, lactoferrin, lactoferricin, and ovispirin such as novispirin (Robert Lehrer, 2000), plectasins, and statins.

Examples of polyunsaturated fatty acids are C₁₈-, C₂₀- and C₂₂-polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Examples of enzyme are phytase (EC 3.1.3.8 or 3.1.3.26), galactanase (EC 3.2.1.89), alpha-galactosidase (EC 3.2.1.22), phospholipase A 1 (EC 3.1.1.32), phospholipase A2 (EC 3.1.1.4), lysophospholipase (EC 3.1.1.5), phospholipase C (EC 3.1.4.3), and/or phospholipase D (EC 3.1.4.4).

Examples of fat-soluble vitamins include but are not limited to vitamin A, vitamin D₃, and vitamin K, e.g. vitamin K₃.

Examples of water-soluble vitamins include but are not limited to vitamin B₁₂, biotin and choline, vitamin B₁, vitamin B₂, vitamin B₆, niacin, folic acid and panthothenate, e.g. Ca-D-panthothenate.

Examples of minerals include but are not limited to calcium, phosphorus, sodium, potassium, magnesium, chlorine, iodine, iron, manganese, copper, molybdenum, cobalt and zinc. Common mineral supplements in feed are: limestone, Bone meal, oyster shell, sodium chloride, dicalcium phosphate, manganese sulphate, potassium iodide, and superphosphate. Sources of minerals include meat scraps, fish meal, milk products, ground limestone (calcium), ground oyster shells (calcium), dicalcium phosphate (calcium, phosphorus), defluorinated rock phosphate (phosphorus, calcium), steamed bone meal (phosphorus, calcium), salt (sodium, chlorine, iodine), manganese sulfate (manganese), manganese oxide (manganese), zinc carbonate (zinc), zinc oxide (zinc).

As also anticipated by any person skilled in the art, the animal feed according to the present invention may further include any number of components typical for an animal feed, such as proteins, carbohydrates as defined above, fats and additional additives.

Examples of suitable types of proteins that can be included in the feed include, but are not limited to, meat scraps (lysine), fish meal (lysine, methionine), poultry by-product meal (tryptophan, lysine), blood meal, liver and glandular meal, feather meal (hydrolyzed), animal tankage, milk products, cottonseed meal, peanut meal, soybean meal, sesame meal, sunflower seed meal.

Most feed ingredients (maize, barley, safflower, milo, wheat, rice, bran, etc.) contain approximately 2-5% fat and linoleic acid. Sources of fats include animal tallow (beef), lard, corn oil, and other vegetable oils.

Additional additives include but are not limited to minerals as defined above; antioxidants like BHT (Butylated hydroxytoluene), santoquin, ethoxyquin, butylated hydroxyanisode and diphenyl paraphenyl diamine; pellet binders such as sodium bentonite (clay), liquid or solid by-products of the wood pulp industry, molasses, and guarmeal; coloring agents such as xanthophylls, synthetic carotinoid, and canthaxanthin; probiotics such as strains of lactobacillus and streptococcus; and/or antibiotics such as penicillin, streptomycin, tetracyclines, and aureomycin.

In one embodiment, the present invention provides a method for boosting the activity of a carbohydrase in an animal feed comprising adding to the animal feed one or more proteolytic enzymes, i.e., proteases, wherein:
a) the protease is an acid stable serine protease, and more preferably a S8 protease, in a dosage of between 10,000 units/kg feed and 30,000 units/kg feed;
b) the carbohydrase is hemicellulase, pectinase, amylase, xylanase or mixture thereof;
c) the animal feed is an animal diet based on soy-bean meal, corn and/or wheat; and
d) optionally the activity of a carbohydrase is increased by 5% or more.

In another embodiment, the present invention provides a method for improving hydrolyzation of a carbohydrate in an animal feed comprising adding to the animal feed one or more proteolytic enzymes, i.e., proteases, and a carbohydrase, wherein:
a) the protease is an acid stable serine protease, and more preferably a S8 protease, in a dosage of between 10,000 units/kg feed and 30,000 units/kg feed;
b) the carbohydrase is hemicellulase, pectinase, amylase, xylanase or mixture thereof;
c) the animal feed is an animal diet based on soy-bean meal, corn and/or wheat; and
d) optionally the activity of a carbohydrase is increased by 5% or more.

In the second aspect, the present invention provides a feed composition comprising one or more protease as defined below and a carbohydrase or carbohydrase mixture.

Proteases of such compositions are:
A: Acid stable proteases selected from the group consisting of:
a) the proteases derived from Nocardiopsis sp. NRRL 18262, and Nocardiopsis alba;
b) proteases of at least 60, 65, 70, 75, 80, 85, 90, or at least 95% amino acid identity to any of the proteases of (i);
c) proteases of at least 60, 65, 70, 75, 80, 85, 90, or at least 95% identity to any of SEQ ID NO: 1, and/or SEQ ID NO: 2.
or
B: Proteases defined by polypeptides having S8 protease activity, wherein the polypeptide is selected from the list consisting of:
a) (a′) a polypeptide having a sequence identity of at least 70% to any one of SEQ ID NOs 3-6;
b) (b′) a variant of any one of SEQ ID NOs: 3-6, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
c) (c′) a polypeptide comprising the polypeptide of (a′) or (b′) and a N-terminal and/or C-terminal His-tag and/or HQ-tag;
d) (d′) a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and
e) (e′) a fragment of the polypeptide of (a′) or (b′) having protease activity and having at least 90% of the length of the mature polypeptide.

The carbohydrase of such compositions may be any carbohydrase which can be added into an animal feed for feeding an animal. Examples of the carbohydrases include but are not limited to hemicellulases, pectinases, glucanases, amylases (such as α-amylase, β-amylase and γ-amylase), xylanases, galactosidases maltases.

Preferred xylanases are 1,4-β-xylanases, for example endo-1,4-β-xylanases produced by Trichoderma reesei. Examples of carbohydrase mixtures are mixtures of different xylanases or mixtures comprising at least two enzymes selected from the group consisting of xylanases, glucanases, hemicellulases, pectinases, amylases, galactosidases, maltases. Mixtures containing a combination of more than 10 active enzymes can be produced by only one non-genetically modified fungus, as for example Talaromyces versatilis.

Commercially available carbohydrases which are covered by the definitions above are Ronozyme®VP (Glucanase, DSM Nutritional Products AG, Switzerland), Ronozyme®WX (Xylanase, DSM Nutritional Products Ltd., Switzerland), RONOZYME® HiStarch (Amylase from Bacillus licheniformis, DSM Nutritional Products AG, Switzerland), Rovabio (Carbohydrase blend, Adisseo, France), ECONASE® (Xylanase, AB Enzymes, Germany), CIBENZA CSM (mixture of xylanase, β-glucanase and galactosidase, Novus International, USA), AllzymeSSF (Carbohydrase blend, Alltech USA).

The feed composition, and/or the components such as the carbohydrase and the protease which the composition contains may be formulated as a liquid formulation or a solid formulation. Accordingly, the feed composition according to the present invention may also comprise one or more formulating agents.

The formulating agents may be selected from the group consisting of polyol such as glycerol, sorbitol, ethylene glycol, diethylene glycol, triethylene glycol, 1,2-propylene glycol, 1,3-propylene glycol, dipropylene glycol and polyethylene glycol (PEG); a salt such as organic or inorganic zinc, sodium, potassium, calcium or magnesium salts (for example, magnesium sulfate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate); and starch or a sugar or sugar derivative such as sucrose, dextrin, glucose, lactose and sorbitol; small organic molecules, flour, cellulose and minerals and clay minerals (also known as hydrous aluminium phyllosilicates such as kaolinite or kaolin).

The feed composition according to the present invention may also comprise one or more emulsifying agents. The emulsifying agents may be selected advantageously from the group consisting of polyglycerol esters of fatty acids such as esterified ricinoleic acid or propylene glycol esters of fatty acids, saccharo-esters or saccharo-glycerides, polyethylene glycol, lecithins, etc.

In the feed composition according to the present invention, the protease may be provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed.

In the feed composition according to the present invention, the carbohydrase may be provided in a dosage of between 10 units/kg animal feed and 5,000 units/kg animal feed, for example in one of the following: 10, 20, 40, 50, 60, 80, 100, 200, 500, 800, 1,000, 2,000, 3,000, 4,000 and 5,000 units/kg animal feed.

As understood by any person skilled in the art, the feed composition according to the present invention may be formulated as a feed additive or an animal feed and thus may further comprise ingredients as defined above suitable for an animal feed.

The third aspect of the present invention provides use of one or more proteolytic enzymes, i.e., proteases, and a carbohydrase in an animal feed for improving digestibility of carbohydrates in an animal, wherein the protease, the carbohydrase, the carbohydrate and the animal feed and the relevant dosage are defined as above.

The present invention will be further illustrated by the following examples.

### EXAMPLES

### Example 1: Compositions Comprising Protease

Compositions 1-3 comprising protease according to the present invention was made by mixing the following ingredients and amounts as shown in Table 1.

TABLE 1

| | Compo- | Compo- | Compo- |
|---|---|---|---|
| | sition 1 | sition 2 | sition 3 |
| ProAct360* | 50 mg | 50 mg | 50 mg |
| RONOZYME ® VP* | 200 mg | | |
| RONOZYME ® WX CT* | | 100 mg | |
| RONOZYME ® HiStarch* | | | 133 mg |

*Trade names of enzyme products commercialized by DSM Nutritional Products AG, Switzerland.

### Example 2: Animal Feed Comprising Protease

An animal feed comprising protease according to the present invention was made by adding protease into the feed with the following formulation as shown in Table 2.

TABLE 2

| | Corn | SBM | Corn/SBM | N-free |
|---|---|---|---|---|
| | diet | diet | diet | diet |
| Ingredient | (g/Kg) | (g/Kg) | (g/Kg) | (g/Kg) |
| Corn | 930 | — | 600 | — |
| Soybean meal | — | 410 | 330 | — |
| Wheat starch | — | 520 | — | 842 |
| Soybean oil | 30 | 30 | 30 | 50 |
| Sodium bicarbonate | 2.0 | 2.0 | 2.0 | 2.0 |
| Sodium chloride | 2.0 | 2.0 | 2.0 | 2.0 |
| Dicalcium phosphate | 19 | 19 | 19 | 19 |
| Limestone | 10 | 10 | 10 | 10 |
| Titanium dioxide | 5.0 | 5.0 | 5.0 | 5.0 |
| Vitamin-mineral premix¹ | 2.0 | 2.0 | 2.0 | 2.0 |
| Solkafloc (Cellulose) | — | — | — | 50 |
| Dipotassium phosphate | — | — | — | 12 |
| RONOZYME ® WX | 200 U/Kg | 200 U/Kg | 200 U/Kg | 200 U/Kg |
| ProAct360 | 15,000 U/Kg | 15,000 U/Kg | 15,000 U/Kg | 15,000 U/Kg |

¹Supplied per kilogram of diet: butylated hydroxy toluene, 100 mg; biotin, 0.2 mg; calcium pantothenate, 12.8 mg; cholecalciferol, 60 μg; cyanocobalamin, 0.017 mg; folic acid, 5.2 mg; menadione, 4 mg; niacin, 35 mg; pyridoxine, 10 mg; trans-retinol, 3.33 mg; riboflavin, 12 mg; thiamine, 3.0 mg; dl-α-tocopheryl acetate, 60 mg; choline chloride, 638 mg; Co, 0.3 mg; Cu, 3.0 mg; Fe, 25 mg; I, 1 mg; Mn, 125 mg; Mo, 0.5 mg; Se, 200 μg; Zn, 60 mg (DSM Nutritional Products, Wagga Wagga, NSW, Australia)

### Example 3: Effect of Protease in the Animal Feed on SBM

The effect of a protease for application in the animal feed on soy-bean meal (SBM) was investigated in an in vitro experiment.

80.1 mg of RONOZYME® VP granules were extracted in 25 ml of 0.1 M acetate buffer+5 mM CaCl₂) PH 6.0. The sample was filtered with a 0.2 μm filter and diluted four times in 0.1 M acetate buffer+5 mM CaCl₂) pH 6.0. 100 μL of the obtained enzyme solution was added to 4 mL of 10% SBM weight/volume. 11.2 μL of a solution of the purified protease (concentration 21.5 mg/mL) was diluted with 1989 μL of 0.1 M acetate buffer+5 mM CaCl₂) pH 6.0. 100 μL of the obtained enzyme solution was added to 4 mL of 10% SBM weight/volume.

Following incubation with the enzymes, the solids were removed by centrifugation for 15 min at 4700 g and 0° C., and 500 μL of the supernatant was subjected to acid hydrolysis (1.6 M HCl) for 1 hour at 99° C. The resulting samples were analyzed for neutral monosaccharide contents by high performance anion exchange chromatography combined with pulsed amperometric detection (HPAEC-PAD). Separation was achieved on a CarboPac analytical PA-210 column (id 2 mm) and a CarboPac PA-210 guard column (Thermofisher) at a temperature of 40° C. with 1 mM KOH isocratic eluent flow rate of 0.2 mL/min.

Same procedures were implemented for the Control group and the RONOZYME® VP group except no enzyme was added in the Control group, and only RONOZYME® VP was added in the RONOZYME® VP group.

Experiments were run in quadruplicates. Six monosaccharide standards (fucose, arabinose, glucose, xylose, mannose and galacturonic acid) were analyzed and the results given as an average of determinations were record in Table 3.

TABLE 3

| | Fucose | Arabinose | Glucose | Xylose | Galacturonic |
|---|---|---|---|---|---|
| | (mg/g | (mg/g | (mg/g | (mg/g | acid (mg/g |
| Treatments | substrate) | substrate) | substrate) | substrate) | substrate) |
| Control | 0.008 | 0.186 | 2.144 | 0.168 | 0.38 |
| RONOZYME ® | 0.014 | 0.248 | 2.334 | 0.262 | 0.35 |
| VP | | | | | |
| RONOZYME ® | 0.020 | 0.256 | 2.392 | 0.398 | 0.49 |
| VP + | | | | | |
| ProAct360 | | | | | |

The experiments demonstrated that RONOZYME® VP breaks down and solubilizes NSPs from SBM, and, when adding protease, 5% more of the NSPs from SBM are hydrolyzed and solubilized.

### Example 4: Use of Protease in the Animal Feed on Destarched Corn

The effect of a protease for application in the animal feed on destarched corn was investigated in an in vitro experiment.

100 mg of RONOZYME® WX CT granules were extracted in 25 mL of 0.1 M acetate buffer+5 mM CaCl₂) PH 6.0. The sample was filtered with a 0.2 μm filter. 100 μL of the obtained enzyme solution was added to 4 mL of 10% destarched corn weight/volume. 11.2 μL of a solution of the purified protease (concentration 21.5 mg/mL) was diluted with 1989 μL of 0.1 M acetate buffer+5 mM CaCl₂) pH 6.0. 100 μL of the obtained enzyme solution was added to 4 mL of 10% destarched corn weight/volume.

Following incubation with the enzymes, the solids were removed by centrifugation for 15 min at 4700 g and 0° C., and 500 μL of the supernatant was subjected to acid hydrolysis (1.6 M HCl) for 1 hour at 99° C. The resulting samples were analyzed for neutral monosaccharide contents by high performance anion exchange chromatography combined with pulsed amperometric detection (HPAEC-PAD). Separation was achieved on a CarboPac analytical PA-210 column (id 2 mm) and a CarboPac PA-210 guard column (Thermofisher) at a temperature of 40° C. with 1 mM KOH isocratic eluent flow rate of 0.2 mL/min.

Same procedures were implemented for the Control group and the RONOZYME® WX group except no enzyme was added in the Control group, and only RONOZYME® WX was added in the RONOZYME® WX group.

Experiments were run in quadruplicates. Arabinose and xylose were analyzed and the results given as an average of determinations were record in Table 4.

TABLE 4

| | Arabinose | Xylose |
|---|---|---|
| Treatments | (mg/g substrate) | (mg/g substrate) |
| Control | 0.037 | 0.036 |
| RONOZYME ® WX | 0.151 | 0.261 |
| RONOZYME ® WX + | 0.157 | 0.346 |
| ProAct360 | | |

The experiments demonstrated that RONOZYME® WX breaks down and solubilizes arabinoxylans from wheat, and, when adding protease, 23% more arabinoxylan is solubilized.

### Example 5: Use of Protease in the Animal Feed on Corn

The effect of a protease for application in the animal feed on corn was investigated in an in vitro experiment.

160.2 mg of RONOZYME® HiStarch granules were extracted in 25 mL of 0.1 M acetate buffer+5 mM CaCl₂) pH 6.0. The sample was filtered with a 0.2 μm filter and diluted eight times in 0.1 M acetate buffer+5 mM CaCl₂) pH 6.0. 100 μL of the obtained enzyme solution was added to 4 mL of 10% corn weight/volume. 11.2 μL of a solution of the purified protease (concentration 21.5 mg/mL) was diluted with 1989 μL of 0.1 M acetate buffer+5 mM CaCl₂) pH 6.0. 100 μL of the obtained enzyme solution was added to 4 mL of 10% corn weight/volume.

Experiments were run in quadruplicates. Following incubation with the enzymes, the solids were removed by centrifugation for 15 min at 4700 g and 0° C., and 500 μl of the supernatant was subjected to acid hydrolysis (1.6 M HCl) for 1 hour at 99° C. The resulting samples were analyzed for neutral monosaccharide contents by high performance anion exchange chromatography combined with pulsed amperometric detection (HPAEC-PAD). Separation was achieved on a CarboPac analytical PA-210 column (id 2 mm) and a CarboPac PA-210 guard column (Thermofisher) at a temperature of 40° C. with 1 mM KOH isocratic eluent flow rate of 0.2 mL/min.

Same procedures were implemented for the Control group and the RONOZYME® HiStarch group except no enzyme was added in the Control group, and only RONOZYME® HiStarch was added in the RONOZYME® HiStarch group.

Experiments were run in quadruplicates. Glucose was analyzed and the results given as an average of determinations were record in Table 5.

TABLE 5

| | Glucose |
|---|---|
| Treatments | (mg/g substrate) |
| Control | 79.09 |
| RONOZYME ® HiStarch | 127.92 |
| RONOZYME ® HiStarch + | 157.87 |
| ProAct360 | |

The experiments demonstrated that RONOZYME® HiStarch hydrolyzes corn starch, and, when adding protease, 21% more corn starch is hydrolyzed.

1. A method for boosting the activity of a carbohydrase in an animal feed comprising adding to the animal feed one or more proteolytic enzymes, i.e., proteases, wherein the protease is a serine protease, preferably an acid stable serine protease or a S8 protease and wherein the carbohydrase is selected from the group consisting of hemicellulase, pectinase, glucanase, amylase, xylanase, maltase and mixture thereof.

2. The method of claim 1, wherein the acid-stable protease is selected from the group consisting of
a. proteases derived from Nocardiopsis sp. NRRL 18262, and Nocardiopsis alba;
b. proteases of at least 60, 65, 70, 75, 80, 85, 90, or at least 95% amino acid identity to any of the proteases of (i);
c. proteases of at least 60, 65, 70, 75, 80, 85, 90, or at least 95% identity to any of SEQ ID NO: 1, and/or SEQ ID NO: 2.

3. The method of claim 1, wherein the protease is defined by polypeptides having S8 protease activity selected from the list consisting of:
a. a polypeptide having a sequence identity of at least 70% to any one of SEQ ID NOs 3-6;
b. a variant of any one of SEQ ID NOs: 3-6, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
c. a polypeptide comprising the polypeptide of (a′) or (b′) and a N-terminal and/or C-terminal His-tag and/or HQ-tag;
d. a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and
e. a fragment of the polypeptide of (a′) or (b′) having protease activity and having at least 90% of the length of the mature polypeptide

4. The method of claim 1, wherein the protease is provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed.

5. The method of claim 1, wherein the carbohydrase is provided in a dosage of between 10 units/kg animal feed and 5,000 units/kg animal feed, for example in one of the following amounts: 10, 20, 40, 50, 60, 80, 100, 200, 500, 800, 1,000, 2,000, 3,000, 4,000 and 5,000 units/kg animal feed.

6. The method of claim 1, wherein the animal feed contains carbohydrates selected from the group consisting of arabinoxylan, starch and/or non-starch polysaccharide (NSP) such as fibre, cellulose, hemicellulose and pectin, or mixture thereof.

7. The method of claim 6, wherein the carbohydrate is in forms of concentrates such as soy-bean meal, corn, wheat, wheat middlings, oats, rye, barley and sorghum; and/or roughages such as hay and pasture plants, or mixture thereof.

8. The method of claim 1, wherein the animal feed is an animal diet based on soy-bean meal, corn and/or wheat.

9. A method for boosting the activity of a carbohydrase in an animal feed comprising adding to the animal feed one or more proteolytic enzymes, i.e., proteases, wherein:
a) the protease is an acid stable serine protease, and more preferably a S8 protease, in a dosage of between 10,000 units/kg feed and 30,000 units/kg feed;
b) the carbohydrase is hemicellulase, pectinase, amylase, xylanase or mixture thereof;
c) the animal feed is an animal diet based on soy-bean meal, corn and/or wheat; and
d) optionally the activity of the carbohydrase is increased by 5% or more.

10. A method for improving hydrolyzation of a carbohydrate in an animal feed comprising adding to the animal feed one or more proteolytic enzymes, i.e., proteases, and a carbohydrase.

11. A method for improving digestibility of a carbohydrate in an animal comprising administering to the animal one or more proteolytic enzymes, i.e., proteases, and a carbohydrase in an animal feed.

12. The method of claim 10, wherein the proteases are a serine protease, preferably an acid stable serine protease, and more preferably a S8 protease.

13. The method of claim 10, wherein the carbohydrase is selected from the group consisting of hemicellulase, pectinase, glucanase, amylase, xylanase, maltase and mixture thereof.

14. A feed composition comprising one or more proteases and one or more carbohydrases, wherein the protease is a serine protease or a S8 protease and wherein the carbohydrase is selected from the group consisting of hemicellulase, pectinase, glucanase, amylase, xylanase, maltase and mixture thereof and wherein
a. the acid-stable protease is selected from the group consisting of
i. proteases derived from Nocardiopsis sp. NRRL 18262, and Nocardiopsis alba;
ii. proteases of at least 60, 65, 70, 75, 80, 85, 90, or at least 95% amino acid identity to any of the proteases of (i);
iii. proteases of at least 60, 65, 70, 75, 80, 85, 90, or at least 95% identity to any of SEQ ID NO: 1, and/or SEQ ID NO: 2; or
b. the protease is defined by polypeptides having S8 protease activity selected from the list consisting of:
i. a polypeptide having a sequence identity of at least 70% to any one of SEQ ID Nos: 3-6;
ii. a variant of any one of SEQ ID NOs: 3-6, wherein the variant has protease activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
iii. a polypeptide comprising the polypeptide of (a′) or (b′) and a N-terminal and/or C-terminal His-tag and/or HQ-tag;
iv. a polypeptide comprising the polypeptide of (a′) or (b′) and a N-terminal and/or C-terminal extension of up to 10 amino acids, e.g. 1, 2, 3, 4, 5, 6, 7, 8, 9 or 10 amino acids; and
v. a fragment of the polypeptide of (a′) or (b′) having protease activity and having at least 90% of the length of the mature polypeptide.
